(19) United States
(12) Patent Application Publication
(10) Pub. No.: US 2022/0047213 A1
(43) Pub. Date: Feb. 17, 2022

(54) Grouping Neuropsychotypes of Patients with Chronic Pain for Personalized Medicine

(71) Applicant: Northwestern University, Evanston, IL (US)

(72) Inventors: Apkar Vania Apkarian, Chicago, IL (US); Etienne Vachon-Presseau, Montreal (CA); Sara E. Berger, Evanston, IL (US); Thomas J. Schnitzer, Chicago, IL (US)

(21) Appl. No.: 17/289,801

(22) PCT Filed: Oct. 28, 2019
(86) PCT No.: PCT/US19/58341

§ 371 Date: Apr. 29, 2021

Related U.S. Application Data
(60) Provisional application No. 62/751,778, filed on Oct. 29, 2018.

(51) Int. Cl. A61B 5/00; A61B 5/16; G16H 10/60; G16H 30/20; G16H 10/20; G16H 50/70; G16H 50/20; G16H 20/70

(52) U.S. Cl. CPC A61B 5/4824; A61B 5/165; A61B 5/4815; G16H 10/60; G01R 33/4806; G16H 10/20; G16H 50/70; G16H 50/20; G16H 20/70; G16H 30/20

(57) ABSTRACT

Systems as described herein can include diagnosing and treating patients with chronic conditions, such as chronic back pain. A transition to chronic pain can include brain adaptations that can carve the state of chronic pain. Additionally, pain characteristics, pain related disability, and responses to treatment can be at least partially determined by psychological factors and/or personality properties. Patients can be classified into a plurality (e.g., five) neuropsychotypes based on set of brain imaging data and psychological assessment. Once classified, personalized treatment options can be developed for chronic pain patients. However, it should be noted that any of a variety of patients with any of a variety of chronic disorders such as, but not limited to, chronic negative mood disorders, such as PTSD, depression, and anxiety, can be diagnosed and treated using any of the systems and processes described herein.

### CROSS-REFERENCE TO RELATED APPLICATIONS

This application claims priority to U.S. Provisional Patent Application No. 62/751,778, titled “Grouping Neuropsychotypes of Patients with Chronic Pain for Personalized Medicine” and filed Oct. 29, 2018, the disclosure of which is hereby incorporated by reference in its entirety.

### FEDERAL FUNDING STATEMENT

This invention was made with government support under AT007987 awarded by the National Institutes of Health. The government has certain rights in the invention.

### FIELD OF DISCLOSURE

This disclosure relates generally to improved pain management and, more particularly, to grouping patients with chronic pain according to neuropsychotypes for personalized medicine.

### BACKGROUND

Pain is associated with negative motions and is highly salient, enabling an organism to escape or protect an injured body part and thereby enhance its chance for survival. However, when pain becomes chronic and the subject must live with the constant pain for many years, the pain becomes maladaptive and modifies the subject's outlook on everyday experiences and future expectations by changing physiological and psychological processes underlying pain perception and pain-related behavior. Brain elements are involved in such processes.

Chronic pain impacts approximately 10% of adults and can occur in the absence of identifiable external stimuli. Chronic pain diminishes quality of life and increases anxiety and depression. Chronic pain can also be associated with cognitive, chemical, and/or morphologic abnormalities. However, there remains a lack of knowledge regarding brain elements involved in such conditions.

### SUMMARY

The following presents a simplified summary of various aspects described herein. This summary is not an extensive overview, and is not intended to identify key or critical elements or to delineate the scope of the claims. The following summary merely presents some concepts in a simplified form as an introductory prelude to the more detailed description provided below. Corresponding apparatus, systems, and computer-readable media are also within the scope of the disclosure.

Systems as described herein can include diagnosing and treating patients with chronic conditions, such as chronic back pain. A transition to chronic pain can include brain adaptations that can carve the state of chronic pain. Additionally, pain characteristics, pain related disability, and responses to treatment can be at least partially determined by psychological factors and/or personality properties. Patients can be classified into a plurality (e.g., five) neuropsychotypes based on set of brain imaging data and psychological assessment. Once classified, personalized treatment options can be developed for chronic pain patients. However, it should be noted that any of a variety of patients with any of a variety of chronic disorders such as, but not limited to, chronic negative mood disorders, such as PTSD, depression, and anxiety, can be diagnosed and treated using any of the systems and processes described herein.

These features, along with many others, are discussed in greater detail below.

### BRIEF DESCRIPTION OF THE DRAWINGS

The present disclosure is described by way of example and not limited in the accompanying figures in which like reference numerals indicate similar elements and in which:

FIG. 1 illustrates a flow diagram of an example method to identify psychological factors and personality traits represented in patterns of brain activity in accordance with at least one aspect of the disclosure;

FIG. 2 illustrates an example neuropsychotype classification system in accordance with at least one aspect of the disclosure;

FIGS. 3A-3B depict example clusters of psychological factors and personality traits in accordance with at least one aspect of the disclosure;

FIGS. 4A-4B show example psychological determinants of chronic pain in accordance with at least one aspect of the disclosure;

FIGS. 5A-5D illustrate that different neuropsychotypes of patients show distinct clinical features in accordance with at least one aspect of the disclosure; and

FIG. 6 is a block diagram of an example processor platform structured to execute instructions in accordance with at least one aspect of the disclosure.

### DETAILED DESCRIPTION

In the following detailed description, reference is made to the accompanying drawings that form a part hereof, and in which is shown by way of illustration specific examples that can be practiced. These examples are described in sufficient detail to enable one skilled in the art to practice the subject matter, and it is to be understood that other examples can be utilized and that logical, mechanical, electrical and other changes can be made without departing from the scope of the subject matter of this disclosure. The following detailed description is, therefore, provided to describe an exemplary implementation and not to be taken as limiting on the scope of the subject matter described in this disclosure. Certain features from different aspects of the following description can be combined to form yet new aspects of the subject matter discussed below.

Brain properties contribute to a risk of developing chronic pain. Additionally, a transition to chronic pain involves brain adaptations that can carve the state of chronic pain. Additionally, pain characteristics, pain related disability, and responses to treatment can be at least partially determined by psychological factors and/or personality properties. Although a biopsychosocial theoretical construct is actively applied in clinical management of chronic pain (e.g., multidisciplinary biopsychosocial rehabilitation for chronic low back pain: Cochrane systematic review and meta-analysis, etc.), component properties that form multidisciplinary biopsychosocial rehabilitation, such as neurobiological processes that determine the chronic pain psychology, remain unknown. Certain examples address this issue by determining whether psychological factors associated with chronic pain are identifiable and whether there are neurobiological underpinnings that can be quantified and reliably linked to the psychology of chronic pain.

Certain examples predict the psychological determinants of chronic pain from resting state functional connectivity (e.g., neurotypes), with the goal of determining if and how psychological factors and personality traits are represented in patterns of brain activity, thus establishing the relationship between psychotypes and neurotypes. Psychological types or psychotypes characterize people in terms of psychological function, such as how they perceive, judge, etc. Neurotypes refer to how the subject's brain is wired. In certain examples, psychotypes and neurotypes exist for chronic pain, and distributed neural representations of psychological determinants of chronic pain are also present. In certain examples, the neurotypes can be used to identify related psychology and establish characteristics of neuropsychotypes, such as for chronic back pain (CBP), etc.

For example, an analysis of an example data set of 62 people suffering from CBP, four psychotypes were identified and validated with respect to an additional 46 CBP sufferers. Two of the four psychotypes related to pain characteristics (e.g., pain and pain-related negative affect). These two psychotypes can be used to identify related biology using resting-state functional connectivity magnetic resonance imaging (rsfcMRI). The two psychotypes can be represented in three specific and dis sociable patterns of functional connectivity, neurotypes. Repeated rsfcMRI sessions conducted in the setting of a randomized controlled trial show that within-subject changes in neurotype expressions mediated the changes seen in pain anxiety and negative affect, suggesting a causal relationship. Furthermore, neurotype pattern expressions can be sufficient to derive five clinically meaningful neuropsychotypes of CBP patients: some neuropsychotypes indicate vulnerability to pain or comorbid mood disorders while others showed resilience or protection. Certain examples demonstrate that psychological determinants of chronic pain have objective neurophysiological representations from which the clinical characteristics of the patients can be inferred.

Thus, using a set of brain imaging data and psychological assessment of patients in chronic pain, patients can be classified into a plurality (e.g., five) neuropsychotypes. The results identify specific questions that can be administered to subjects with pain, and, based on the results, one can identify clinically useful subtypes. Once classified, personalized treatment options can be developed for chronic pain patients, for example. However, it should be noted that any of a variety of patients with any of a variety of chronic disorders such as, but not limited to, chronic negative mood disorders, such as PTSD, depression, and anxiety, can be diagnosed and treated using any of the systems and processes described herein.

For example, rsfcMRI images can be interrogated to identify (e.g., using a 3-fold cross-validation procedure) stable (across scans) brain functional connectivity network biotypes for the two pain-related psychotypes. These biotypes mediated changes between anxiety of pain to changes in negative affective qualities and segregated CBP to 5 neuropsychotypes with distinct pain characteristics, independent of pain duration or intensity. Thus, psychological determinants of chronic pain can be neurobiological contributors to CBP pathology. Neuropsychotypes of chronic pain can be established based on these psychological determinants.

In certain examples, answers to a set of questions are obtained from a chronic pain patient, and, based on these answers, a score is calculated. Based on the score, the particular patient is characterized into one of five distinct biopsychotypes. These subtypes have utility in clinical decision making and in personalized medicine since each subtype shows a differential treatment response. Based on the classified psychotypes, treatment plans can be determined for the patients and these treatment plans can be administered to the patients.

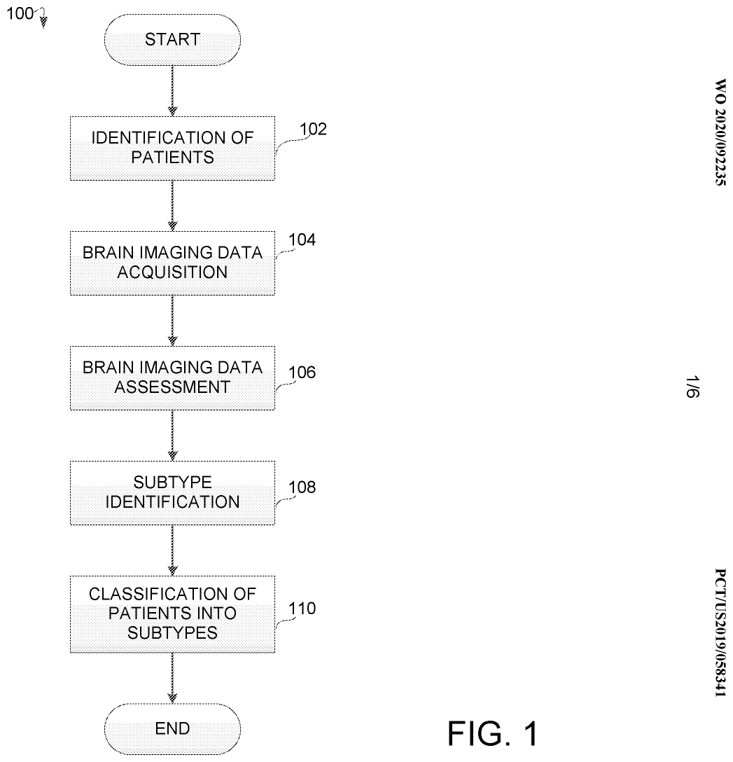

FIG. 1 illustrates a flow diagram of an example method 100 to identify psychological factors and personality traits represented in patterns of brain activity of chronic back pain (CBP) patients in order to establish relationship between psychotypes (PTs) and neurotypes (NTs). At block 102, a set or cohort of CBP patients is identified for data collection purposes. The set of patients is identified and organized to identify and validate psychotypes related to pain characteristics.

At block 104, brain imaging data acquisition is performed for each of the patients in the group. For example, brain imaging data can be acquired using resting-state functional connectivity MRI (rsfcMRI) to determine PTs represented in specific patterns of functional connectivity NTs. Randomized controlled trials can be used to further elucidate NT effects on pain anxiety and/or other pain state and determine within-subject changes in NT expression.

At block 106, NT expression-based identification of CBP patient neuropsychotypes is performed. Expression-based identification of CBP patient neuropsychotypes can include self-reported condition data provided by the patient according to a variety of questionnaires and techniques as described herein. In other examples, other parameters such as sleep disturbance, ability to do daily chores, social interactions, mobility or exercise, etc., can be identified based on data assessment.

At block 108, subtype identification is attained. In one example, five clinically meaningful neuropsychotypes are obtained through the identification of pain outcomes and related psychological properties. In several embodiments, patients are classified into subtypes based on scores calculated using a weighted function of the brain imaging data and/or self-reported test results. Example equations 1-5 used to derive these PTs based on pain related measures are shown below, with normalizations of previously established parameters into categories (e.g. high, medium, low):

Neuropsychotype 1=MPQs(high)+MPQa(high)+PainDetect(high)+PANASn(high)+BDI(medium)+SF12(medium) (Eq. 1);

Neuropsychotype 2=MPQs(medium)+MPQa(medium)+PainDetect(low)+PANASn(high)+BDI(high)+SF12(low) (Eq. 2);

Neuropsychotype 3=Memory(high)+MPQs(low)+MPQa(low)+PainDetect(low)+PANASn(low)+BDI(low)+SF12(medium) (Eq. 3);

Neuropsychotype 4=MPQs(medium)+MPQa(medium)+PainDetect(medium)+PANASn(medium)+BDI(medium)+SF12(medium) (Eq. 4);

Neuropsychotype 5=MPQs(medium)+MPQa(low)+PainDetect(medium)+PANASn(medium)+BDI(medium)+SF12(high) (Eq. 5).

Functions used in Equations 1-5 include McGill Pain Questionnaire (MPQ) sensory and affective scales (e.g., a high score reflects a worse degree of pain), PainDetect screening questionnaire to identify neuropathic components in patients, positive and negative affect schedule (PANAS) self-reporting questionnaire, Beck Depression Inventory (BDI) psychometric test to measure severity of depression, and SF-12 health questionnaire to categorize mental and physical functioning and overall health-related quality of life.

At block 110, resulting subtype identification enables classification of CBP patients into a specific subtype to differentiate clinical treatments and responses to treatment for these patients. These subtypes enable clinical decision making that drives personalized medicine, given that treatment response variations can be identified by knowing within which category or subtype the patient belongs.

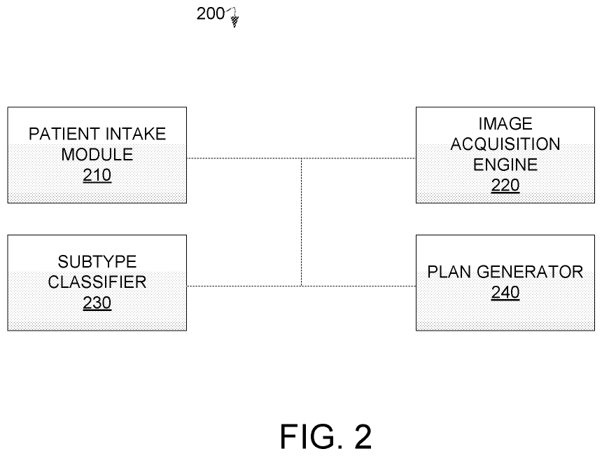

FIG. 2 illustrates an example neuropsychotype classification system 200 including a patient intake module 210 to process patient records and identify patients for analysis. The example system 200 includes an image acquisition engine 220 to acquire brain imaging data for the patient(s) and process the acquired imaging data. The example system 200 includes a subtype classifier 230 to process patient data and brain imaging data, as well as associated analysis, to classify the patient according to a neuropsychotype. The example system includes a plan generator 240 to generate a plan (e.g., treatment plan, etc.), report, and/or other recommendation for the patient(s) based on the type classification and additional patient and reference data.

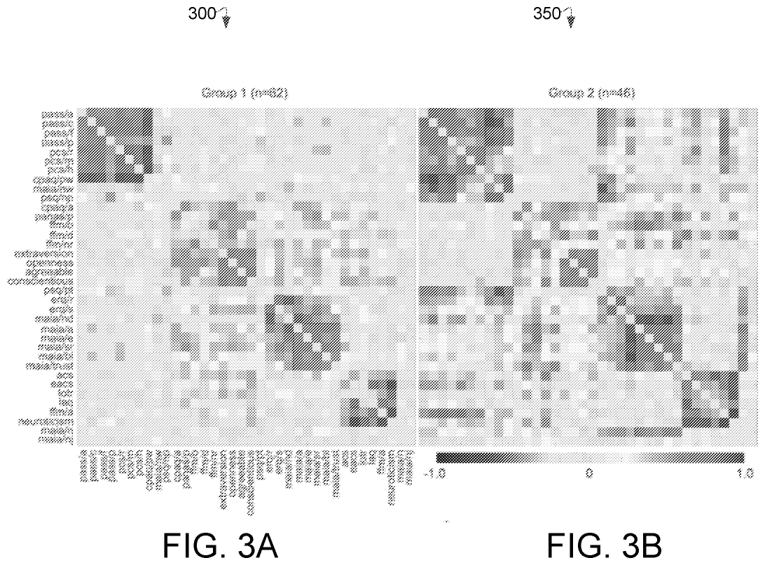

FIGS. 3A-3B depict example clusters of psychological factors and personality traits clustering to four psychotypes. As illustrated in the covariance matrix 300 of FIG. 3A, associations are shown between questionnaire subscales that were used to probe CBP patients' psychological profiles, one for each CBP group. In the example of FIG. 3B, principal component analysis (PCA) was performed on all 37 questionnaire subscales administered to patients in Group 1 350 and identified 4 relevant components: pain-related psychotype (Pain-Psyche 1); interoceptive traits (Intero-Psyche 2); self-awareness abilities (Aware-Psyche 3); and emotional stability (Emote-Psyche 4). PCA performed on the same variables in the group 2 patients identified the same 4 components (validation).

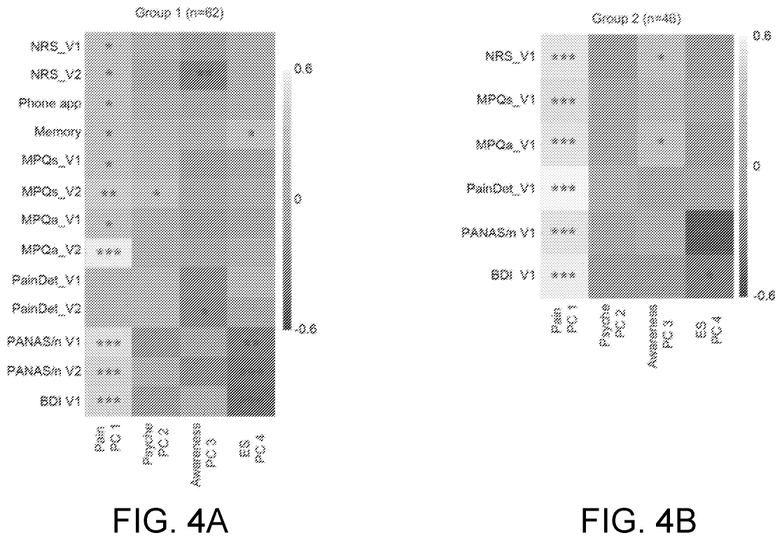

FIGS. 4A-4B show example psychological determinants of chronic pain. In group 1 of FIG. 4A, Pain-psyche 1 scores were related to almost all pain characteristics queried and emote-psyche 4 scores were negatively related to negative affect. As demonstrated in FIG. 4B, consistent findings were observed in group 2. *p<0.05; **p<0.01; ***p<0.001.

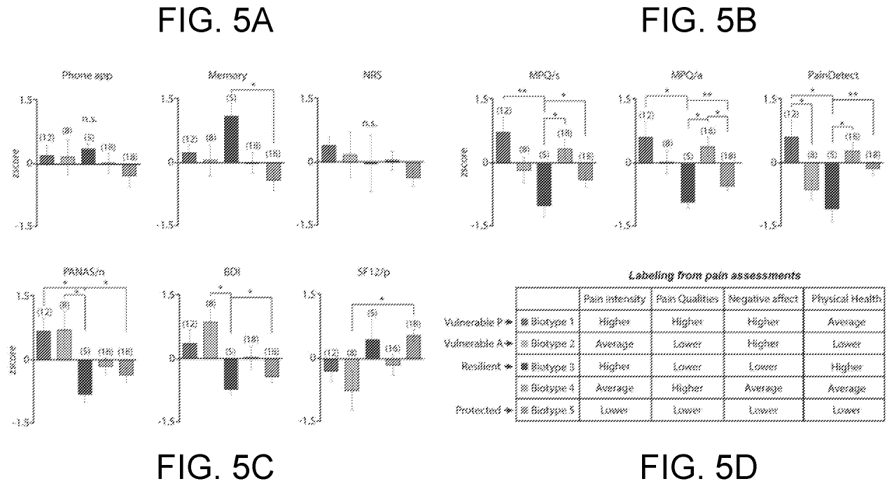

FIGS. 5A-5D illustrate that different neuropsychotypes of CBP patients show distinct clinical features. A clinical profile of each neuropsychotype can be assessed based on nine pain and affect related outcome measures. As illustrated in FIG. 5A, there are no group differences for pain intensity measured by the phone app and the numeric rating scale (NRS), but the neuropsychotypes differ in their verbal recall of pain experienced over the last week (memory). In the example of FIG. 5B, groups differed in pain qualities measured with the MPQa, MPQs, and PainDetect. In the example of FIG. 5C, groups also differed on negative affect measured with the PANAS/n and on depression (BDI), as well as on a measurement of physical health (SF12/p). In the example of FIG. 5D, each neuropsychotype was labeled based on correspondence with the mean of the group across the nine clinical outcomes: Neuropsychotype 1 shows vulnerability to Pain; Neuropsychotype 2 shows vulnerability to negative affect; Neuropsychotype 3 shows resilience; Neuropsychotype 4 tended to reflect the average of the outcome measures; and Neuropsychotype 5 shows protection. (Neuropsychotypes are indicated as Biotypes in d).

While example implementations are illustrated in conjunction with FIGS. 1-5D, elements, processes and/or devices illustrated in conjunction with FIGS. 1-5D can be combined, divided, re-arranged, omitted, eliminated and/or implemented in any other way. Further, components disclosed and described herein can be implemented by hardware, machine readable instructions, software, firmware and/or any combination of hardware, machine readable instructions, software and/or firmware. Thus, for example, components disclosed and described herein can be implemented by analog and/or digital circuit(s), logic circuit(s), programmable processor(s), application specific integrated circuit(s) (ASIC(s)), programmable logic device(s) (PLD(s)) and/or field programmable logic device(s) (FPLD(s)). When reading any of the apparatus or system claims of this patent to cover a purely software and/or firmware implementation, at least one of the components is/are hereby expressly defined to include a tangible computer readable storage device or storage disk such as a memory, a digital versatile disk (DVD), a compact disk (CD), a Blu-ray disk, etc. storing the software and/or firmware.

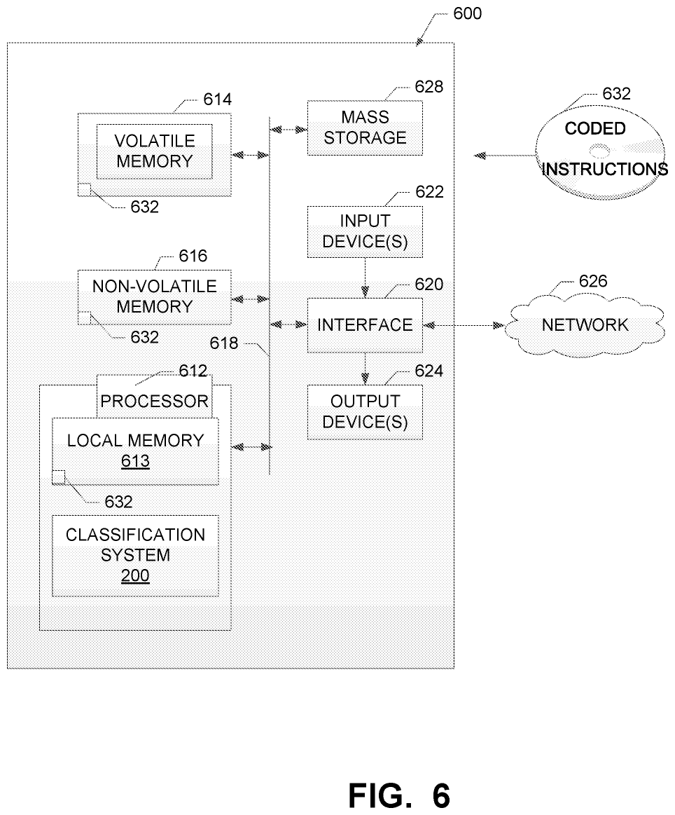

A flowchart representative of example machine readable instructions for implementing components disclosed and described herein is shown in conjunction with at least FIG. 1. In the examples, the machine/computer readable instructions include a program for execution by a processor such as the processor 612 shown in the example processor platform 600 discussed below in connection with FIG. 6. The program can be embodied in machine readable instructions stored on a tangible computer readable storage medium such as a CD-ROM, a floppy disk, a hard drive, a digital versatile disk (DVD), a Blu-ray disk, or a memory associated with the processor 612, but the entire program and/or parts thereof could alternatively be executed by a device other than the processor 612 and/or embodied in firmware or dedicated hardware. Further, although the example program is described with reference to the flowcharts illustrated in conjunction with at least FIG. 1, many other methods of implementing the components disclosed and described herein can alternatively be used. For example, the order of execution of the blocks can be changed, and/or some of the blocks described can be changed, eliminated, or combined. Although the flowchart of at least FIG. 1 depicts example operations in an illustrated order, these operations are not exhaustive and are not limited to the illustrated order. In addition, various changes and modifications can be made by one skilled in the art within the spirit and scope of the disclosure. For example, blocks illustrated in the flowchart can be performed in an alternative order or can be performed in parallel.

As mentioned above, the example process(es) of at least FIG. 1 can be implemented using coded instructions (e.g., computer and/or machine readable instructions) stored on a tangible computer readable storage medium such as a hard disk drive, a flash memory, a read-only memory (ROM), a compact disk (CD), a digital versatile disk (DVD), a cache, a random-access memory (RAM) and/or any other storage device or storage disk in which information is stored for any duration (e.g., for extended time periods, permanently, for brief instances, for temporarily buffering, and/or for caching of the information). As used herein, the term tangible computer readable storage medium is expressly defined to include any type of computer readable storage device and/or storage disk and to exclude propagating signals and to exclude transmission media. As used herein, “tangible computer readable storage medium” and “tangible machine-readable storage medium” are used interchangeably. Additionally or alternatively, the example process(es) of at least FIG. 1 can be implemented using coded instructions (e.g., computer and/or machine readable instructions) stored on a non-transitory computer and/or machine readable medium such as a hard disk drive, a flash memory, a read-only memory, a compact disk, a digital versatile disk, a cache, a random-access memory and/or any other storage device or storage disk in which information is stored for any duration (e.g., for extended time periods, permanently, for brief instances, for temporarily buffering, and/or for caching of the information). As used herein, the term non-transitory computer readable medium is expressly defined to include any type of computer readable storage device and/or storage disk and to exclude propagating signals and to exclude transmission media. As used herein, when the phrase “at least” is used as the transition term in a preamble of a claim, it is open-ended in the same manner as the term “comprising” is open ended. In addition, the term “including” is open-ended in the same manner as the term “comprising” is open-ended.

FIG. 6 is a block diagram of an example processor platform 600 structured to executing the instructions of at least FIG. 1 to implement the example components disclosed and described herein with respect to FIGS. 2-5D. The processor platform 600 can be, for example, a server, a personal computer, a mobile device (e.g., a cell phone, a smart phone, a tablet such as an iPad™), a personal digital assistant (PDA), an Internet appliance, or any other type of computing device.

The processor platform 600 of the illustrated example includes a processor 612. The processor 612 of the illustrated example is hardware. For example, the processor 612 can be implemented by integrated circuits, logic circuits, microprocessors or controllers from any desired family or manufacturer.

The processor 612 of the illustrated example includes a local memory 613 (e.g., a cache). The example processor 612 of FIG. 6 executes the instructions of at least FIG. 1 to implement the systems and infrastructure and associated methods of FIGS. 1-5D such as the example neuropsychotype classification system 200, etc. The processor 612 of the illustrated example is in communication with a main memory including a volatile memory 614 and a non-volatile memory 616 via a bus 618. The volatile memory 614 can be implemented by Synchronous Dynamic Random Access Memory (SDRAM), Dynamic Random Access Memory (DRAM), RAMBUS Dynamic Random Access Memory (RDRAM) and/or any other type of random access memory device. The non-volatile memory 616 can be implemented by flash memory and/or any other desired type of memory device. Access to the main memory 614, 616 is controlled by a clock controller.

The processor platform 600 of the illustrated example also includes an interface circuit 620. The interface circuit 620 can be implemented by any type of interface standard, such as an Ethernet interface, a universal serial bus (USB), and/or a PCI express interface.

In the illustrated example, one or more input devices 622 are connected to the interface circuit 620. The input device(s) 622 permit(s) a user to enter data and commands into the processor 612. The input device(s) can be implemented by, for example, a sensor, a microphone, a camera (still or video), a keyboard, a button, a mouse, a touchscreen, a track-pad, a trackball, isopoint and/or a voice recognition system.

One or more output devices 624 are also connected to the interface circuit 620 of the illustrated example. The output devices 624 can be implemented, for example, by display devices (e.g., a light emitting diode (LED), an organic light emitting diode (OLED), a liquid crystal display, a cathode ray tube display (CRT), a touchscreen, a tactile output device, and/or speakers). The interface circuit 620 of the illustrated example, thus, typically includes a graphics driver card, a graphics driver chip or a graphics driver processor.

The interface circuit 620 of the illustrated example also includes a communication device such as a transmitter, a receiver, a transceiver, a modem and/or network interface card to facilitate exchange of data with external machines (e.g., computing devices of any kind) via a network 626 (e.g., an Ethernet connection, a digital subscriber line (DSL), a telephone line, coaxial cable, a cellular telephone system, etc.).

The processor platform 600 of the illustrated example also includes one or more mass storage devices 628 for storing software and/or data. Examples of such mass storage devices 628 include floppy disk drives, hard drive disks, compact disk drives, Blu-ray disk drives, RAID systems, and digital versatile disk (DVD) drives.

The coded instructions 632 of FIG. 6 can be stored in the mass storage device 628, in the volatile memory 614, in the non-volatile memory 616, and/or on a removable tangible computer readable storage medium such as a CD or DVD.

From the foregoing, it will be appreciated that the above disclosed methods, apparatus, and articles of manufacture have been disclosed to improve the functioning of a computer and/or computing device and its interaction with image and other patient data for patient classification and personalized medicine.

Thus, certain examples determine categorizations and leverage those calculations to help determine what kind of treatments should be given to which kind of patients. A combination of analysis of patient psychology with image data enables categorization of patients. Brain images are clustered according to certain patterns and linked with psychological profiles. Machine learning instructs the linkage between the psychological profile with the image clusters. In certain examples, brain images are used to identify categories, and, once categories are identified, the brain image signatures are no longer needed. The categories become a universal signature so that brain images are not needed after the categories are established.

Certain examples provide multi-dimensional characterization using both holistic and diagnostic categories of a patient. Patients can include high risk patients and low risk patients. High risk patients can better respond to certain treatments, while low risk patients can respond to exercise therapy, etc. Categories can be related to treatment outcomes, how well category patients handle their treatment, get better, etc. A comprehensive psychological profile can be developed using machine learning and/or other artificial intelligence, for example.

One or more aspects discussed herein can be embodied in computer-usable or readable data and/or computer-executable instructions, such as in one or more program modules, executed by one or more computers or other devices as described herein. Generally, program modules include routines, programs, objects, components, data structures, and the like. that perform particular tasks or implement particular abstract data types when executed by a processor in a computer or other device. The modules can be written in a source code programming language that is subsequently compiled for execution, or can be written in a scripting language such as (but not limited to) HTML or XML. The computer executable instructions can be stored on a computer readable medium such as a hard disk, optical disk, removable storage media, solid-state memory, RAM, and the like. As will be appreciated by one of skill in the art, the functionality of the program modules can be combined or distributed as desired in various embodiments. In addition, the functionality can be embodied in whole or in part in firmware or hardware equivalents such as integrated circuits, field programmable gate arrays (FPGA), and the like. Particular data structures can be used to more effectively implement one or more aspects discussed herein, and such data structures are contemplated within the scope of computer executable instructions and computer-usable data described herein. Various aspects discussed herein can be embodied as a method, a computing device, a system, and/or a computer program product.

Although the present invention has been described in certain specific aspects, many additional modifications and variations would be apparent to those skilled in the art. In particular, any of the various processes described above can be performed in alternative sequences and/or in parallel (on different computing devices) in order to achieve similar results in a manner that is more appropriate to the requirements of a specific application. It is therefore to be understood that the present invention can be practiced otherwise than specifically described without departing from the scope and spirit of the present invention. Thus, embodiments of the present invention should be considered in all respects as illustrative and not restrictive. Accordingly, the scope of the invention should be determined not by the embodiments illustrated, but by the appended claims and their equivalents.

1. A method, comprising:
obtaining, by a processor platform, a first patient data of a first group of patients indicating a suffering from a chronic condition;
obtaining, by the processor platform, brain imaging data associated with the first group of patients;
obtaining, by the processing platform, self-reported condition data from each patient of the first group of patients, indicating an effect of the chronic condition of each patient of the first group of patients;
classifying, by the processing platform, the-each patient into at least one neuropsychotype subtype based on the brain imaging data, and the self-reported condition data of the each patient;
determining, by the processing platform, a first set of treatment plans based on the classifications; and
administering the first set of treatment plans to a second group of patients based on indication of chronic conditions of the each of the second group of patients.

2. The method of claim 1, further comprising obtaining the brain imaging data using a resting-state functional MRI device.

3. The method of claim 1, wherein the brain imaging data comprises psychotypes represented in specific patterns of functional connectivity neurotypes.

4. The method of any of claim 1, wherein:
the self-reported condition data comprises a plurality of self-assessment test results; and
classifying the patient into a neuropsychotype subtype comprises calculating a score using a weighted function of the plurality of self-assessment test results.

5. The method of claim 4, wherein the self-assessment test results comprise results selected from the group consisting of a McGill Pain Questionnaire response, a PainDetect screening response, a positive and negative affect schedule questionnaire response, a beck depression inventory psychometric test response, and a SF-12 health questionnaire response.

6. The method of any of claim 1, wherein the self-reported condition data comprises conditions selected from the group consisting of sleep disturbances, ability to perform daily chores, social interactions, mobility issues, and exercise issues.

7. The method of any of claim 1, further comprising randomly selecting, by the processing platform, the patient data from a cohort of patients.

8. A processing platform, comprising:
a processor; and
a memory storing instructions that, when executed by the processor, cause the processing platform to:
obtain a first patient data of a first group of patients indicating a suffering from a chronic condition;
obtain brain imaging data associated with the first group of patients;
obtain self-reported condition data from each patient of the first group of patients, indicating an effect of the chronic condition of each patient of the first group of patients;
classify each patient into at least one neuropsychotype subtype based on the brain imaging data, and the self-reported condition data of each patient;
determine a first set of treatment plans based on the classifications; and
administer the first set of treatment plans to a second group of patients based on indication of chronic conditions of the each of the second group of patients.

9. The processing platform of claim 8, wherein the instructions, when executed by the processor, further cause the processing platform to obtain the brain imaging data using a resting-state functional MRI device.

10. The processing platform of claim 8, wherein the brain imaging data comprises psychotypes represented in specific patterns of functional connectivity neurotypes.

11. The processing platform of claim 8, wherein:
the self-reported condition data comprises a plurality of self-assessment test results; and
the instructions, when executed by the processor, further cause the processing platform to classify the patient into a neuropsychotype subtype by calculating a score using a weighted function of the plurality of self-assessment test results.

12. The processing platform of claim 11, wherein the self-assessment test results comprises results selected from the group consisting of a McGill Pain Questionnaire response, a PainDetect screening response, a positive and negative affect schedule questionnaire response, a beck depression inventory psychometric test response, and a SF-12 health questionnaire response.

13. The processing platform of claim 8, wherein the self-reported condition data comprises conditions selected from the group consisting of sleep disturbances, ability to perform daily chores, social interactions, mobility issues, and exercise issues.

14. The processing platform of claim 8, wherein the instructions, when executed by the processor, further cause the processing platform to randomly select the patient data from a cohort of patients.

15. A non-transitory machine-readable medium storing instructions that, when executed by one or more processors, cause the one or more processors to perform steps comprising:
obtaining a first patient data of a first group of patients indicating a suffering from a chronic condition;
obtaining brain imaging data associated with the first group of patients;
obtaining self-reported condition data from each patient of the first group of patients, indicating an effect of the chronic condition of each patient of the first group of patients;
classifying each patient into at least one neuropsychotype subtype based on the brain imaging data, and the self-reported condition data of each patient;
determining a first set of treatment plans based on the classifications; and
administering the first set of treatment plans to a second group of patients based on indication of chronic conditions of the each of the second group of patients.

16. The non-transitory machine-readable medium of claim 15, wherein the instructions, when executed by one or more processors, further cause the one or more processors to perform steps comprising obtaining the brain imaging data using a resting-state functional MRI device.

17. The non-transitory machine-readable medium of claim 15, wherein the brain imaging data comprises psychotypes represented in specific patterns of functional connectivity neurotypes.

18. The non-transitory machine-readable medium of claim 15, wherein:
the self-reported condition data comprises a plurality of self-assessment test results; and
the instructions, when executed by one or more processors, further cause the one or more processors to classify the patient into a neuropsychotype subtype by calculating a score using a weighted function of the plurality of self-assessment test results.

19. The non-transitory machine-readable medium of claim 18, wherein the self-assessment test results comprises results selected from the group consisting of a McGill Pain Questionnaire response, a PainDetect screening response, a positive and negative affect schedule questionnaire response, a beck depression inventory psychometric test response, and a SF-12 health questionnaire response.

20. The non-transitory machine-readable medium of claim 15, wherein the self-reported condition data comprises conditions selected from the group consisting of sleep disturbances, ability to perform daily chores, social interactions, mobility issues, and exercise issues.
